(19) United States
(12) Patent Application Publication
(10) Pub. No.: US 2025/0058149 A1
(43) Pub. Date: Feb. 20, 2025

(54) HYBRID BEAMFORMING DEVICE FOR ULTRASOUND THERAPY, AND OPERATING METHOD THEREFOR

(71) Applicant: MUTI Inc., Daejeon (KR)

(72) Inventor: Kyung Il Cho, Daejeon (KR)

(73) Assignee: MUTI Inc., Daejeon (KR)

(21) Appl. No.: 18/721,400

(22) PCT Filed: Feb. 24, 2022
(86) PCT No.: PCT/KR2022/002715

§ 371 Date: Jun. 18, 2024

(30) Foreign Application Priority Data
Dec. 22, 2021 (KR) 10-2021-0184539

(51) Int. Cl. A61N 7/00; B06B 1/06

(52) U.S. Cl. CPC A61N 7/00; B06B 1/0625; B06B 1/0651; A61N 2007/0026; A61N 2007/0056; B06B 2201/76

(57) ABSTRACT

The present invention relates to a hybrid beamforming device for ultrasound therapy, and an operating method therefor, the device using tile chips, which include circularly arranged semiconductor ultrasound chips, so as to be capable of modular beam focusing, and comprising: tile chips including circularly arranged semiconductor ultrasound chips formed from a plurality of annular array channels, and a control unit, which links with multi-focusing of annular arrays in the depth direction caused by beamforming of the tile chips, so as to control the position of the tile chips, and thus focus, at a target point, ultrasound beams generated at the tile chips.

### TECHNICAL FIELD

The present invention relates to a hybrid beamforming device of ultrasound therapy and an operating method thereof, and more particularly, to a hybrid beamforming device capable of performing modular beam focusing using a tile chip in which semiconductor ultrasound chips are arranged in a circular shape.

### RELATED ART

When used in a focused manner, ultrasound is safe and may apply high energy to deep areas of a human body.

The existing mechanical piezoelectric (PZT) focused ultrasound is a single channel-based fixed focusing method using a concave lens and has a limited application. Therefore, although there is a great interest in a high-performance PZT two-dimensional (2D) array, more than 10,000 transducer channels and beamforming are required for multi-depth focusing of the PZT 2D array and there is a limitation that manufacturing cost of micro-processing and beamformers is very expensive.

Meanwhile, semiconductor ultrasound may perform multi-focusing in a two-dimensional (2D) array and may dramatically reduce unit cost through a wafer processing, so much research is being conducted at advanced research institutes on multi-depth focusing therapy. In particular, therapy using an annular array single chip of semiconductor ultrasound is being widely studied since it is simple to perform focused beamforming control with only a few channels, but has the disadvantage that a focused stimulation intensity is limited due to weaker generation pressure compared to the existing commercial ultrasound. To resolve this, the advanced research institute allows a semiconductor ultrasound transducer cell array to perform modular integration for a plurality of chips with a regular matrix in a tile form and to simultaneously focus the same through beamforming to increase output intensity. However, this mainly requires the user of hybrid beamforming (frontend analog beamform×backend digital beamforming) for electronic beamforming of 10,000 or more transducer channels, which has the limitation of being very difficult and expensive.

### DETAILED DESCRIPTION

### Technical Subject

An objective of the present invention is to perform custom-focusing of hybrid beamforming through multi-channel beamforming without high-difficulty hybrid beamforming in using a plurality of tile chips to increase output that is a disadvantage of semiconductor ultrasound. Therefore, the present invention is to implement custom-focusing using a tile chip in which semiconductor ultrasound chips formed with a plurality of annular array channels are arranged in a circular shape.

However, technical subjects to be solved by the present invention are not limited to the aforementioned subjects and may be variously expanded without departing from the technical spirit and scope of the present invention.

### Solution

A hybrid beamforming device of ultrasound therapy according to an example embodiment of the present invention includes a tile chip including semiconductor ultrasound chips formed with a plurality of annular array channels and arranged in a circular shape; and a controller configured to control a position of the tile chip in conjunction with depth direction multi-focusing of an annular array by beamforming of the tile chip to focus ultrasound beams generated from the tile chip at a target point.

Also, the hybrid beamforming device according to an example embodiment of the present invention may further include a receiver provided as a reception chip at the center of the tile chip and configured to receive reflected pulse echo of a signal generated from the circularly arranged tile chip.

The receiver may be configured to perform at least one of a calibration function by pulse echo reception for modular transmission of the tile chip and a function of detecting the fascia.

The tile chip may include at least one semiconductor ultrasound chip provided in a tile-shaped circular modular structure and at least one of corners of the tile chip may have a truncated shape.

The semiconductor ultrasound chip may include a micro-machined ultrasonic transducer (MUT) array formed of a plurality of vibration elements arranged in an annular shape and configured to apply a low frequency or a high frequency depending on the application depth inside the skin, and the MUT array may include a parallel connection wiring between upper electrodes configured for each channel of the vibration element and a connection wiring between lower electrodes provided in a longitudinal direction.

A receiver may be present at the center of an annular shape of each semiconductor ultrasound chip and the ground of a transmitter and the receiver may be separated, making transmission and reception on a single chip possible.

The MUT array may include a plurality of channels in which the plurality of vibration elements are arranged in a preset number, and each channel may have vibration elements arranged in at least two rows.

The controller may be configured to control the position of the tile chip linked with the depth direction multi-focusing of the annular array by beamforming of the tile chip using a reception signal through the pulse echo received by the receiver to custom-focus the ultrasound beams generated from the tile chip on the target point.

The controller may be configured to simultaneously adjust electronic depth direction beamforming of the tile chip and an angle control of the tile chip and to implement depth-specific focusing by adjusting the at least one semiconductor ultrasound chip as a single chip.

The controller may be configured to implement a position control by adjusting an angle or a radial position of the tile chip.

The controller may be configured to implement a position control of the tile chip through a micro linkage and an actuator.

The controller may be configured to simplify and thereby implement a position control of the tile chip through a micromachined metal etching plate structure.

The hybrid beamforming device according to an example embodiment of the present invention may configured to provide an optimized contact on the curved surface, including room temperature vulcanization (RTV) on a front portion in which the tile chip is provided.

The hybrid beamforming device according to an example embodiment of the present invention may be configured to provide a customized stimulation to the fascia and muscles of a human body through multi-focusing using reflected waves of a modular signal from the fascia, muscles, and bones.

The hybrid beamforming device according to an example embodiment of the present invention may be configured to penetrate the skull of a head and to stimulate the brain through multi-focusing. Here, the hybrid beamforming device may be configured to penetrate the skull using a frequency range of 500 kHz to 1 MHz, considering semiconductor ultrasound characteristics.

An operating method of a hybrid beamforming device of ultrasound therapy according to an example embodiment of the present invention includes transmitting ultrasound beams using a tile chip including semiconductor ultrasound chips formed of a plurality of annular array channels and arranged in a circular shape; receiving reflected pulse echo through a receiver positioned at the center of the tile chip; and controlling a position and an angle of the tile chip linked with depth direction multi-focusing of an annular array by beamforming of the tile chip using a reception signal to perform custom-focusing of the ultrasound beams generated from the tile chip on a target point.

### Effect

According to example embodiments of the present invention, in using a plurality of tile chips to increase output that is a disadvantage of semiconductor ultrasound, it is possible to perform custom-focusing of hybrid beamforming through multi-channel beamforming without high-difficulty hybrid beamforming. Therefore, the present invention may perform custom-focusing using a tile chip in which semiconductor ultrasound chips formed with a plurality of annular array channels are arranged in a circular shape.

More specifically, the present invention may easily implement depth-specific focusing as if controlling a single chip by synchronizing control of a tile chip in which semiconductor ultrasound chips formed with a multi-channel annular array are arranged in a tile-shaped circular modular structure and then linking electronic depth direction beamforming and position control (chip angle) of the tile chip and by simultaneously adjusting beamforming and position control of the tile chip.

However, the effects of the present invention are not limited to the aforementioned effects and may be variously expanded without departing from the technical spirit and scope of the present invention.

### BRIEF DESCRIPTION OF DRAWINGS

FIG. 1 is a block diagram illustrating a detailed configuration of a hybrid beamforming device according to an example embodiment of the present invention.

FIG. 2 illustrates hybrid focus adjustment.

FIG. 3 illustrates an example of a front portion including a tile chip and a receiver of a hybrid beamforming device according to an example embodiment of the present invention.

FIGS. 4A to 4C illustrate detailed examples of a semiconductor ultrasound chip according to an example embodiment of the present invention.

FIGS. 5 to 10 illustrate examples of describing hybrid beam focusing through adjustment of a location and an angle of a tile chip according to an example embodiment of the present invention.

FIGS. 11 and 12 illustrate application examples of a hybrid beamforming device according to an example embodiment of the present invention.

FIG. 13 is a flowchart illustrating an operating method of a hybrid beamforming device according to an example embodiment of the present invention.

### BEST MODE

Advantages and features of the present invention and methods to achieve the same will become clear with reference to example embodiments described in detail with the accompanying drawings. However, the present invention is not construed as being limited to the example embodiments disclosed below and will be implemented in various forms different from each other. The example embodiments are provided to make the disclosure of the present invention complete and to inform the scope of the present invention to one of ordinary skill in the art to which the present invention pertains and the present invention is only defined by the scope of the claims.

The terminology used herein is for the purpose of describing the example embodiments only and is not to be limiting the present invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated components steps, operations, and/or elements, but do not preclude the presence or addition of one or more other components, steps, operations, and/or elements.

Unless otherwise defined herein, all terms used herein (including technical or scientific terms) have the same meanings as those generally understood by one of ordinary skill in the art. Also, terms defined in dictionaries generally used should be construed to have meanings matching contextual meanings in the related art and are not to be construed as an ideal or excessively formal meaning unless otherwise defined herein.

Hereinafter, example embodiments of the present invention will be described in detail with reference to the accompanying drawings. Like reference numerals refer to like components throughout and repeated description related thereto will be omitted.

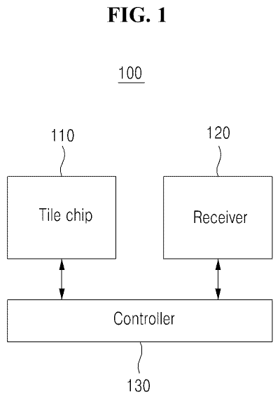

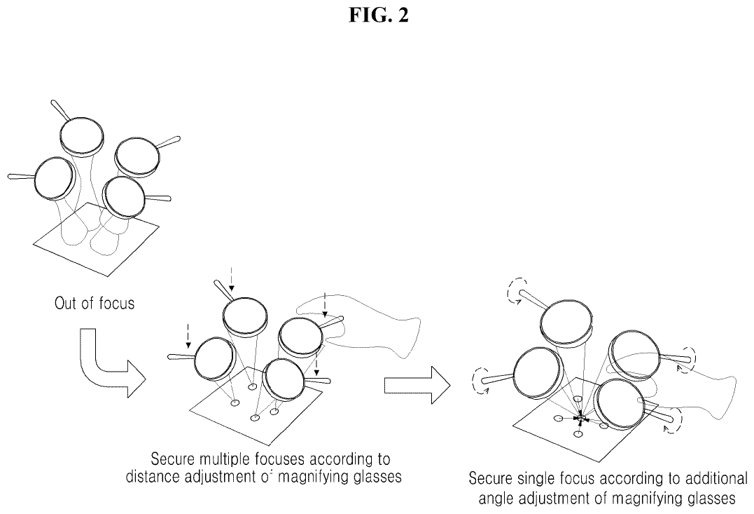

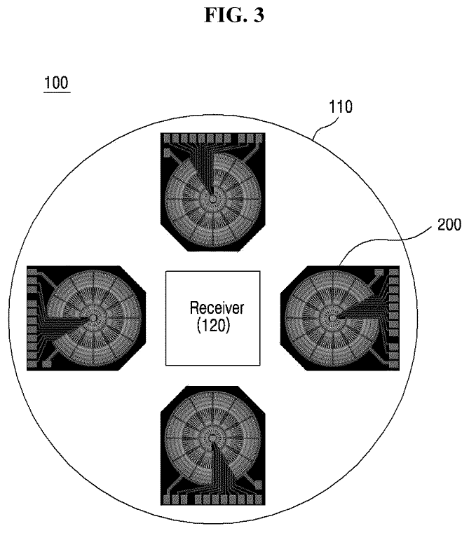

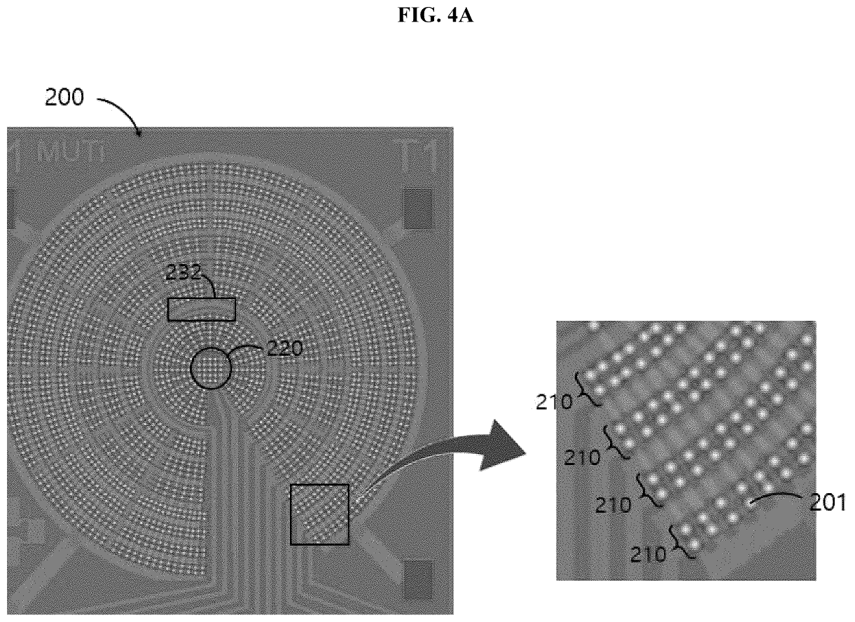

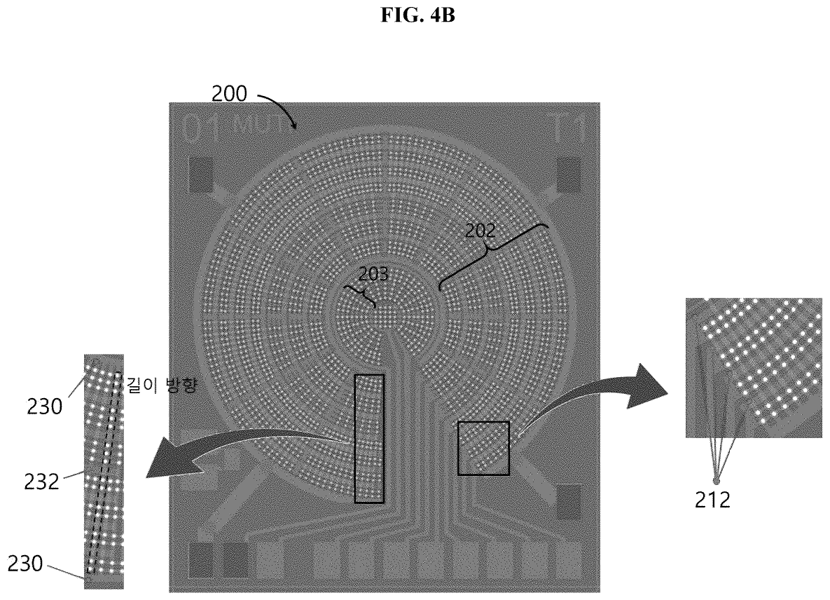

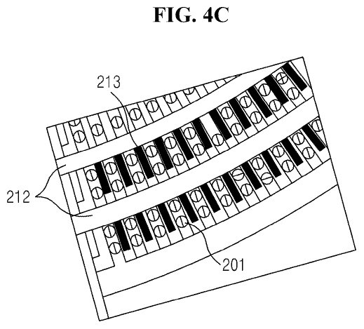

FIG. 1 is a block diagram illustrating a detailed configuration of a hybrid beamforming device according to an example embodiment of the present invention. Also, FIG. 2 illustrates hybrid focus adjustment. Also, FIG. 3 illustrates an example of a front portion including a tile chip and a receiver of a hybrid beamforming device according to an example embodiment of the present invention, and FIGS. 4A to 4C illustrate detailed examples of a semiconductor ultrasound chip according to an example embodiment of the present invention.

The hybrid beamforming device according to an example embodiment of the present invention shown in FIG. 1 may perform modular beam focusing using a tile chip in which semiconductor ultrasound chips are arranged in a circular shape.

To this end, a hybrid beamforming device 100 according to an example embodiment of the present invention includes a tile chip 110, a receiver 120, and a controller 130. The hybrid beamforming device 100 according to an example embodiment of the present invention is to perform custom-focusing of hybrid beamforming with multi-channel beamforming without high-difficulty hybrid beamforming in using a plurality of tile chips to increase output that is a disadvantage of semiconductor ultrasound.

Referring to FIG. 2, to perform beam focusing using a plurality of magnifying glasses, a plurality of focuses according to distance adjustment of the magnifying glasses needs to be secured and a single focus according to additional angle adjustment of the magnifying glasses needs to be secured. Through this, hybrid focus adjustment may be performed. Using this principle, the hybrid beamforming device 100 according to an example embodiment of the present invention may perform custom-focusing of hybrid beamforming through position and angle control of a tile chip configured by arranging at least one semiconductor ultrasound chip (magnifying glass in FIG. 2).

The tile chip 110 includes semiconductor ultrasound chips formed with a plurality of annular array channels and arranged in a circular shape. In the present invention, the tile chip 110 is named including circularly arranged at least one semiconductor ultrasound chip 200 and each tile chip 110 may refer to the semiconductor ultrasound chip 200. The semiconductor ultrasound chip 200 does not necessarily need to have a square shape and, as shown in FIG. 2, at least one of corners may have a truncated shape.

Describing the tile chip 110 with reference to FIG. 3, the tile chip 110 is provided on a front portion that is positioned in front of the hybrid beamforming device 100 and contacts a portion of the body, and the tile chip 110 may include the at least one semiconductor ultrasound chip 200 arranged in a tile-shaped circular modular structure. Although FIG. 3 illustrates four semiconductor ultrasound chips 200, the number and arrangement of semiconductor ultrasound chips 200 are not limited thereto.

The semiconductor ultrasound chip 200 may include a micro-machined ultrasonic transducer (MUT) array (or 200) formed of a plurality of vibration elements arranged in an annular shape and configured to apply a low frequency or a high frequency depending on the application depth inside the skin. Here, the MUT array may include a parallel connection wiring between upper electrodes configured for each channel of the vibration element and a connection wiring between lower electrodes provided in a longitudinal direction. Also, the MUT array may include a plurality of channels in which the plurality of vibration elements are arranged in a preset number, and each channel may have vibration elements arranged in at least two rows.

Describing the semiconductor ultrasound chip 200 with reference to FIGS. 4A to 4C, in FIG. 4A, a plurality of vibration elements (or transmitters) 201 are arranged at regular intervals and form a channel 210, and a plurality of channels 210 are arranged in an annular shape in the MUT array 200. Depending on example embodiments, each channel 210 may include vibration elements 201 arranged in at least two rows, but the number of vibration elements 201 that form the corresponding channel 210 is not limited thereto.

Upper electrodes 211 and 221 and lower electrodes 230 and 231 are connected to each of the plurality of channels 210 formed with the vibration elements 201. In FIGS. 4A and 4B, the upper electrode 211 for transmission is connected to the channel 210 provided at a first location 202 and a second location 203 of the MUT array and the upper electrode 221 is connected to a receiver 220 positioned as an independent channel at the center of the MUT array. Here, a low frequency vibration element is provided at the first location 202 and a high frequency vibration element is provided at the second location 203. The receiver 220 positioned at the center of the annular shape of each semiconductor ultrasound chip 200 has the ground separate from the transmitter. Therefore, transmission and reception may be performed with a single chip. As shown in FIG. 3, the receiver 220 may also be mounted at the center of the tile chip 110.

Also, the lower electrodes include annular connection wirings 230 and 231 on the outer side and the inner side of the MUT array and on the outer side of the center. Therefore, the present invention may minimize disturbance of high-amplification reception signals of a transmission ground portion by separating the ground of multi-channel transmission of transmission elements that are low-frequency vibration elements formed at the first location 202 and reception of the receiver 220 as shown in a box 232.

Referring to FIGS. 4B and 4C, in connection wiring between upper electrodes of the vibration elements 201, a reference connection wiring 212 by the upper electrodes 211 and 221 is provided in an annular shape between the channels 210. The present invention minimizes signal voltage drop along a longitudinal direction by adding a horizontally arranged (or series arranged) electrode wiring, that is, a parallel connection wiring 213 between upper electrodes in the reference connection wiring 212 based on the reference connection wiring 212.

In a connection wiring between lower electrodes corresponding to upper electrodes of the vibration elements 201, a connecting wiring 232 between lower electrodes is provided in a radial direction (or longitudinal direction) to intersect the reference connection wiring 212 between the upper electrodes provided in the annular shape between the respective channels 210. The present invention includes the annular connection wirings 230 and 231 on the outer side and the inner side of the MUT array and on the outer side of the center. For example, the first annular connecting wiring 230 is provided in the annular shape on the outer side and the inner side of the channels 210 provided at the first position 202, and the second annular connection wiring 231 is provided in the annular shape on the outer side of the channels 210 arranged at the second position 203. Therefore, the present invention minimizes voltage drop between lower electrodes.

Referring again to FIG. 1, the receiver 120 of the hybrid beamforming device 100 according to an example embodiment of the present invention is provided as a reception chip at the center of the tile chip 110 and may receive reflected pulse echo of a signal generated from the circularly arranged tile chip 110.

As shown in FIG. 3, the receiver 120 is positioned at the center of the tile chip 110 and the at least one semiconductor ultrasound chip 200 is circularly arranged at regular intervals based on the receiver 120. Therefore, the receiver 120 may detect that at least one of a calibration function by pulse echo reception for modular transmission of the semiconductor ultrasound chip 200 and a function of detecting the fascia is performed, and may provide customized stimulation by multi-focusing transmission accordingly. For example, when a signal transmitted through a transmission element, which refers to vibration elements connected to an upper electrode for transmission, is reflected at a specific position of the subcutaneous or the fascia inside the skin, the receiver 120 may receive a reception signal of reflected pulse echo.

The controller 130 of the hybrid beamforming device 100 according to an example embodiment of the present invention controls a position of the tile chip 110 in conjunction with depth-direction multi-focusing of the annular array by beamforming of the semiconductor ultrasound chip 200 to focus ultrasound beams generated from the tile chip 110 at a target point.

The controller 130 may control the position of the tile chip 110 linked with the depth direction multi-focusing of the annular array by beamforming of the semiconductor ultrasound chip 200 using a reception signal of pulse echo received by the receiver 120 to perform custom-focusing of the ultrasound beams generated from the tile chip 110 on the target point. Also, the controller 130 may simultaneously adjust electronic depth direction beamforming of the tile chip 110 and angle control of the tile chip 110 and may implement depth-specific focusing by adjusting the at least one semiconductor ultrasound chip 200 as a single chip. Here, the controller 130 may implement position control by adjusting an angle or a radial position of the tile chip 110. That is, the controller 130 may provide customized stimulation by multi-focusing transmission by controlling a position and an angle of the tile chip 110 by calibration function and fascia detection using the reception signal through the pulse echo received by the receiver 120 and by controlling ultrasound beam transmission of the tile chip 110.

Here, the controller 130 according to an example embodiment of the present invention may implement position control of the tile chip 110 through a micro linkage and an actuator and may simplify and implement position control of the tile chip 110 through a micromachined metal etching plate structure.

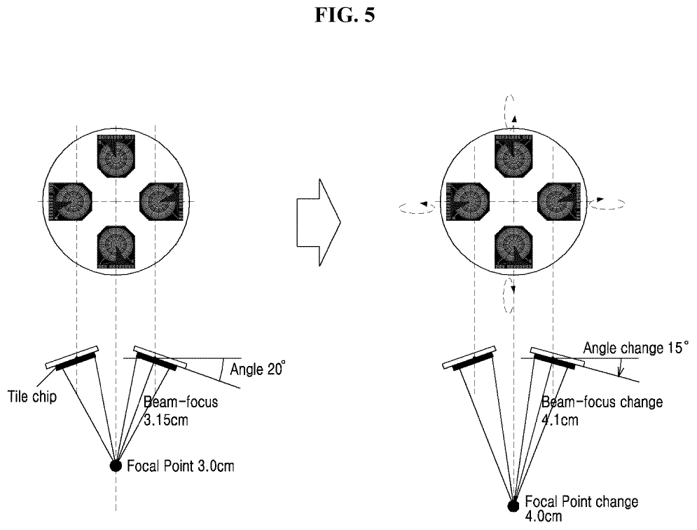

FIGS. 5 to 10 illustrate examples of describing hybrid beam focusing through adjustment of a location and an angle of a tile chip according to an example embodiment of the present invention.

FIG. 5 illustrates an example of describing hybrid beamforming according to a focal point of a tile chip of semiconductor ultrasound.

Referring to FIG. 5, a hybrid beamforming device according to an example embodiment of the present invention may perform modular beam focusing using a tile chip in which four semiconductor ultrasound chips of an annular array are annularly arranged in a tile shape. Here, if a focus point is 3.0 cm, an electronic beam focus is 3.15 cm and a mechanical tile angle is 20 degrees, and if a focus point is 4.0 cm, an electronic beam focus is 4.10 cm and a mechanical tile angle is 15 degrees. Accordingly, hybrid beam focusing may occur due to electronic annular array beam focusing of each semiconductor ultrasound chip and mechanical chip arrangement angle adjustment of the semiconductor ultrasound chip (hybrid beam focusing=electronic annular array beam focusing×mechanical chip arrangement angle adjustment).

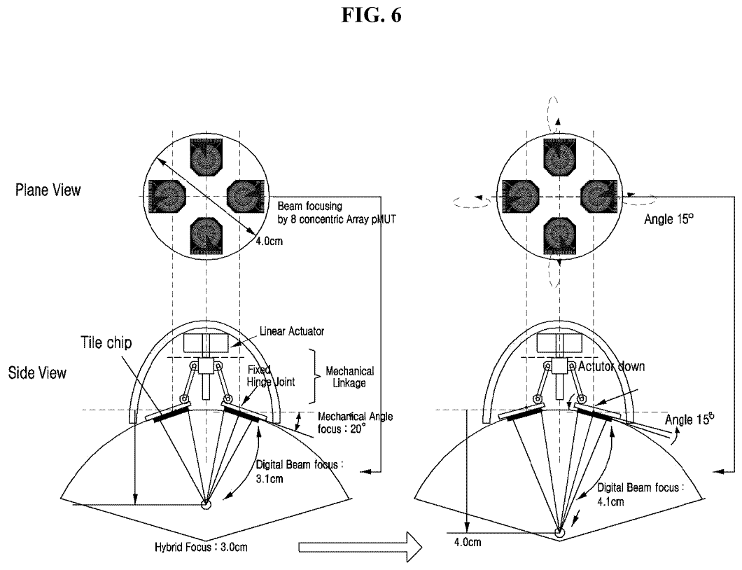

FIG. 6 illustrates an operating mechanism for the mechanical angle adjustment of the semiconductor ultrasound chip shown in FIG. 5. In (a) of FIG. 6, a semiconductor ultrasound chip exhibits a beam focus of about 3.0 cm. However, in (b) of FIG. 6, the semiconductor ultrasound chip with an adjusted angle of 15 degrees exhibits a beam focus of about 4.0 cm. When a central actuator of a tile module moves upward, an edge hinge portion of a tile chip is lifted and an angle of a central hinge portion of the tile chip changes accordingly. Therefore, it can be seen that custom-focusing of hybrid beamforming is possible due to angle adjustment of the semiconductor ultrasound chip.

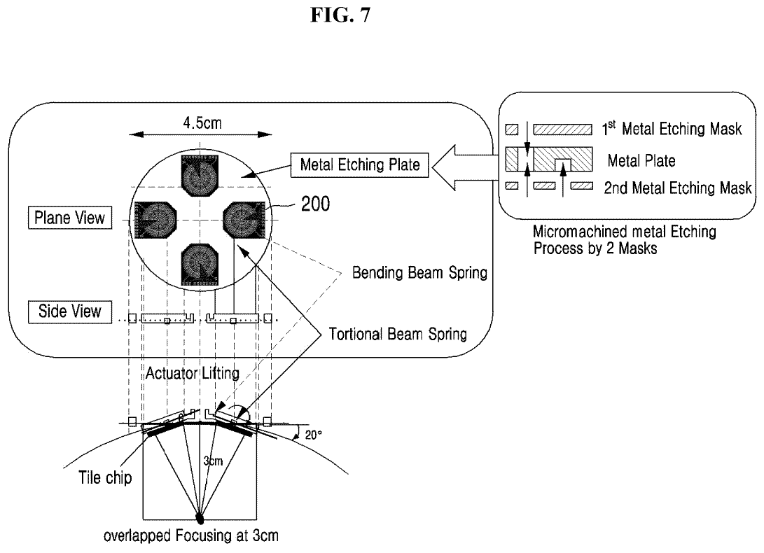

FIG. 7 illustrates an operating mechanism for mechanical angle adjustment by a micromachined metal etching plate structure. Referring to FIG. 7, a plate of a tile chip in which at least one semiconductor ultrasound chip is arranged shows a micromachined metal etching plate structure configured by including a first metal etching mask, a second metal etching mask, and a metal plate between metal etching masks. This is implemented as a thin metal plate using metal etching by replacing two rotation hinge portions in the angle adjustment mechanism of FIG. 6 with a bending beam and a torsion beam, respectively. The present invention may simplify and implement position control of the tile chip by adjusting an angle through the micromachined metal etching plate structure that contacts a portion of the body or the skin.

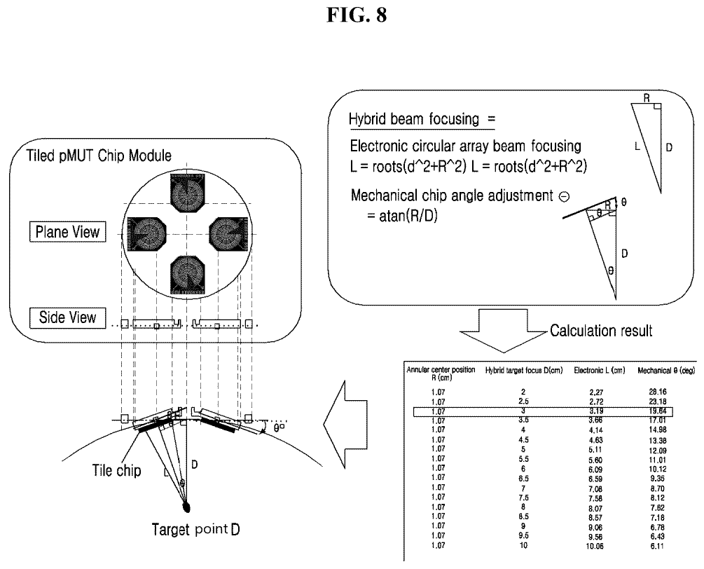

FIG. 8 illustrates formula of calculating an electronic beam distance and a mechanical angle of hybrid beamforming in a semiconductor ultrasound tile chip. Referring to FIG. 8, hybrid beam focusing may be implemented due to the operating mechanism for mechanical angle adjustment by the micromachined metal etching plate structure shown in FIG. 7 and hybrid beam focusing for target point D may occur due to electronic annular array beam focusing of each semiconductor ultrasound chip and mechanical chip arrangement angle adjustment of the semiconductor ultrasound chip.

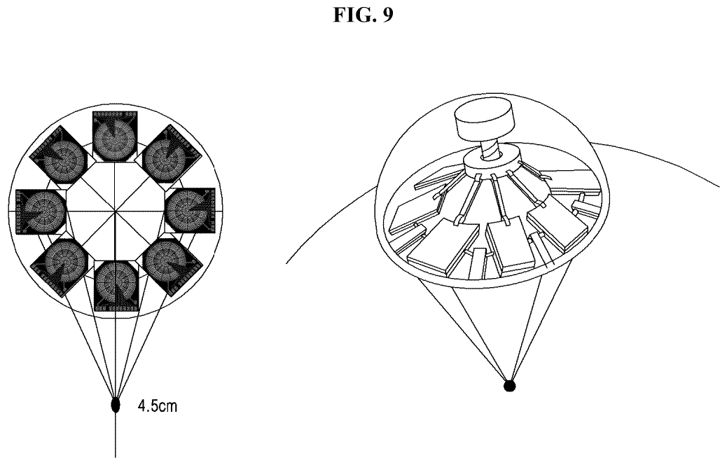

Referring to FIG. 9, a hybrid beamforming device according to an example embodiment of the present invention may perform modular beam focusing by arranging eight semiconductor ultrasound chips in an annular shape to increase the ultrasound power of focus. Semiconductor ultrasound chips formed on a tile chip may be symmetrically arranged and ultrasound beams with the increased power may be provided according to an increase in the number thereof.

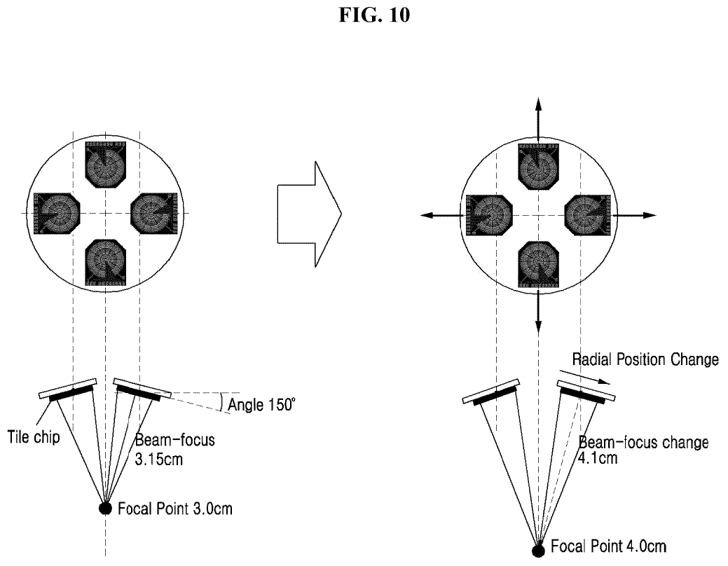

FIG. 10 illustrates hybrid beamforming of a semiconductor ultrasound tile chip implemented with radial direction position adjustment instead of angle adjustment of a mechanical chip. Instead of the mechanical angle adjustment shown in FIG. 5, modular beam focusing may be performed using a tile chip in which four semiconductor ultrasound chips of an annular array are annularly arranged in a tile form due to radial direction position adjustment.

Accordingly, hybrid beam focusing may occur due to electronic annular array beam focusing of each semiconductor ultrasound chip and mechanical radial direction chip position adjustment of the semiconductor ultrasound chip (hybrid beam focusing=electronic annular array beam focusing×mechanical radial direction chip position adjustment).

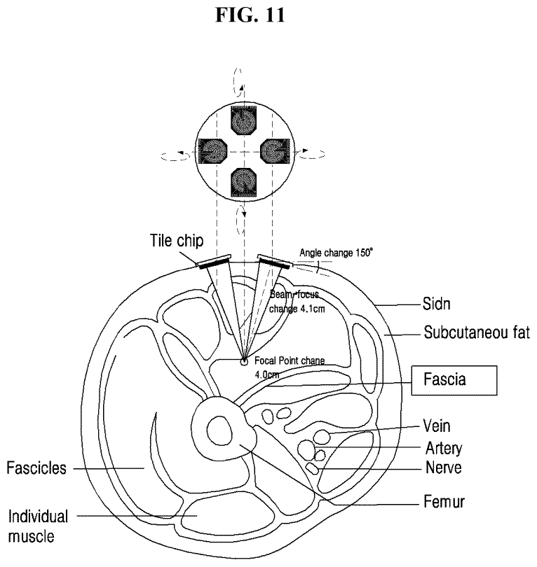

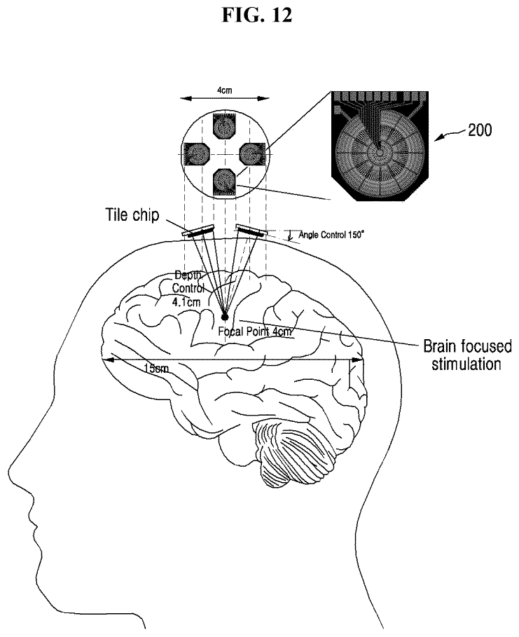

FIGS. 11 and 12 illustrate application examples of a hybrid beamforming device according to an example embodiment of the present invention.

The hybrid beamforming device according to an example embodiment of the present invention is to provide an optimized contact on the curved surface, including room temperature vulcanization (RTV) on a front portion in which a tile chip is provided.

Accordingly, as shown in FIG. 11, the hybrid beamforming device according to an example embodiment of the present invention may provide customized stimulation to the fascia, muscles, and bones present deep in the thigh through hybrid beamforming of the semiconductor ultrasound tile chip 200.

Also, as shown in FIG. 12, the hybrid beamforming device according to an example embodiment of the present invention may penetrate the skull of a head through hybrid beamforming of the semiconductor ultrasound tile chip 200, to stimulate the brain through multi-focusing, and to penetrate the skull using a frequency range of 500 kHz to 1 MHz, considering semiconductor ultrasound characteristics.

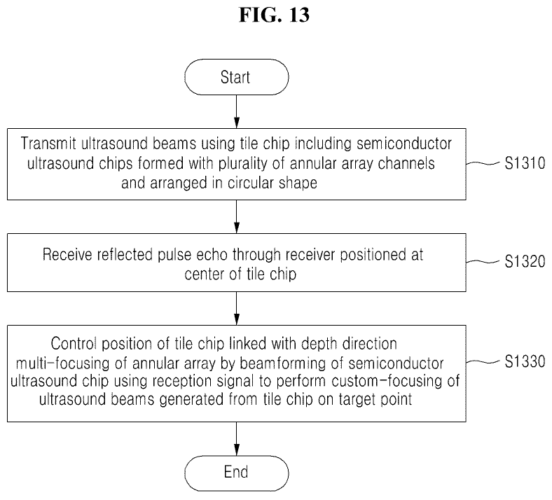

FIG. 13 is a flowchart illustrating an operating method of a hybrid beamforming device according to an example embodiment of the present invention.

The method of FIG. 13 represents a process of operating the hybrid beamforming device according to an example embodiment of the present invention according to a transmission focus auto calibration function.

Referring to FIG. 13, in operation S1310, ultrasound beams are transmitted using a tile chip including semiconductor ultrasound chips formed of a plurality of annular array channels and arranged in a circular shape.

In operation S1320, reflected pulse echo is received through a receiver positioned at the center of the tile chip.

In operation S1330, a position and an angle of the tile chip linked with depth direction multi-focusing of an annular array by beamforming of the semiconductor ultrasound tile chip 110 is controlled using a reception signal to perform custom-focusing of the ultrasound beams generated from the tile chip on a target point. Operation S1330 may perform custom-focusing on the target point by measuring an arrival time using a reception signal received at a specific position of the body different for each individual, by calculating a distance by dividing the arrival time by a speed, and by adjusting a position and an angle of the tile chip including the semiconductor ultrasound chip to stimulate the fascia and muscles of the body at the specific position or the brain based on the calculated distance value.

The systems or the apparatuses described herein may be implemented using hardware components, software components, and/or combinations of the hardware components and the software components. For example, the apparatuses and the components described herein may be implemented using one or more general-purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will be appreciated that a processing device may include multiple processing elements and/or multiple types of processing elements. For example, a processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be permanently or temporarily embodied in any type of machine, component, physical equipment, virtual equipment, a computer storage medium or device, or a signal wave to be transmitted, to be interpreted by the processing device or to provide an instruction or data to the processing device. The software also may be distributed over network coupled computer systems so that the software is stored and executed in a distributed fashion. The software and data may be stored by one or more computer readable storage media.

The methods according to the example embodiments may be configured in a form of program instructions performed through various computer devices and recorded in computer-readable media. The media may also include, alone or in combination with the program instructions, data files, data structures, and the like. The program instructions recorded in the media may be specially designed and configured for the example embodiments or may be known to those skilled in the computer software art and thereby available. Examples of the media include magnetic media such as hard disks, floppy disks, and magnetic tapes; optical media such as CD-ROM and DVDs; magneto-optical media such as floptical disks; and hardware devices that are specially configured to store program instructions, such as read-only memory (ROM), random access memory (RAM), flash memory, and the like. Examples of program instructions include both machine code, such as produced by a compiler, and files containing higher level code that may be executed by the computer using an interpreter. The hardware device may be configured to operate as at least one software module, or vice versa.

### Mode

While the example embodiments are described with reference to specific example embodiments and drawings, it will be apparent to one of ordinary skill in the art that various alterations and modifications in form and details may be made in these example embodiments without departing from the spirit and scope of the claims and their equivalents. For example, suitable results may be achieved if the described techniques are performed in a different order and/or if components in a described system, architecture, device, or circuit are combined in a different manner, or replaced or supplemented by other components or their equivalents.

Therefore, other implementations, other example embodiments, and equivalents of the claims are to be construed as being included in the claims.

What is claimed is:

1. A hybrid beamforming device of ultrasound therapy, the hybrid beamforming device comprising:
a tile chip including semiconductor ultrasound chips formed with a plurality of annular array channels and arranged in a circular shape; and
a controller configured to control a position of the tile chip in conjunction with depth direction multi-focusing of an annular array by beamforming of the tile chip to focus ultrasound beams generated from the tile chip at a target point.

2. The hybrid beamforming device of claim 1, further comprising:
a receiver provided as a reception chip at the center of the tile chip and configured to receive reflected pulse echo of a signal generated from the circularly arranged tile chip.

3. The hybrid beamforming device of claim 2, wherein the receiver is configured to perform at least one of a calibration function by pulse echo reception for modular transmission of the tile chip and a function of detecting the fascia.

4. The hybrid beamforming device of claim 1, wherein the tile chip includes at least one semiconductor ultrasound chip provided in a tile-shaped circular modular structure and at least one of corners of the tile chip has a truncated shape.

5. The hybrid beamforming device of claim 1, wherein the semiconductor ultrasound chip includes a micro-machined ultrasonic transducer (MUT) array formed of a plurality of vibration elements arranged in an annular shape and configured to apply a low frequency or a high frequency depending on the application depth inside the skin, and
the MUT array includes a parallel connection wiring between upper electrodes configured for each channel of the vibration element and a connection wiring between lower electrodes provided in a longitudinal direction.

6. The hybrid beamforming device of claim 5, wherein a receiver is present at the center of an annular shape of each semiconductor ultrasound chip and the ground of a transmitter and the receiver is separated, making transmission and reception on a single chip possible.

7. The hybrid beamforming device of claim 5, wherein the MUT array includes a plurality of channels in which the plurality of vibration elements are arranged in a preset number, and each channel has vibration elements arranged in at least two rows.

8. The hybrid beamforming device of claim 2, wherein the controller is configured to control the position of the tile chip linked with the depth direction multi-focusing of the annular array by beamforming of the tile chip using a reception signal through the pulse echo received by the receiver to perform custom-focusing of the ultrasound beams generated from the tile chip on the target point.

9. The hybrid beamforming device of claim 1, wherein the controller is configured to simultaneously adjust electronic depth direction beamforming of the tile chip and an angle control of the tile chip and to implement depth-specific focusing by adjusting the at least one semiconductor ultrasound chip as a single chip.

10. The hybrid beamforming device of claim 9, wherein the controller is configured to implement a position control by adjusting an angle or a radial position of the tile chip.

11. The hybrid beamforming device of claim 9, wherein the controller is configured to implement a position control of the tile chip through a micro linkage and an actuator.

12. The hybrid beamforming device of claim 9, wherein the controller is configured to simplify and thereby implement a position control of the tile chip through a micromachined metal etching plate structure.

13. The hybrid beamforming device of claim 1, wherein the hybrid beamforming device is configured to provide an optimized contact on the curved surface, including room temperature vulcanization (RTV) on a front portion in which the tile chip is provided.

14. The hybrid beamforming device of claim 2, wherein the hybrid beamforming device is configured to provide a customized stimulation to the fascia and muscles of a human body through multi-focusing using reflected waves of a modular signal from the fascia, muscles, and bones.

15. The hybrid beamforming device of claim 1, wherein the hybrid beamforming device is configured to penetrate the skull of a head and to stimulate the brain through multi-focusing.

16. The hybrid beamforming device of claim 15, wherein the hybrid beamforming device is configured to penetrate the skull using a frequency range of 500 kHz to 1 MHz, considering semiconductor ultrasound characteristics.

17. An operating method of a hybrid beamforming device of ultrasound therapy, the method comprising:
transmitting ultrasound beams using a tile chip including semiconductor ultrasound chips formed of a plurality of annular array channels and arranged in a circular shape;
receiving reflected pulse echo through a receiver positioned at the center of the tile chip; and
controlling a position and an angle of the tile chip linked with depth direction multi-focusing of an annular array by beamforming of the tile chip using a reception signal to perform custom-focusing of the ultrasound beams generated from the tile chip on a target point.
